(19) United States
(12) Patent Application Publication
(10) Pub. No.: US 2025/0361548 A1
(43) Pub. Date: Nov. 27, 2025

(54) ENHANCED SMALL MOLECULE DETECTION AND QUANTITATIVE ANALYSIS

(71) Applicant: Aveta.Life Inc., Hoboken, NJ (US)

(72) Inventors: Natasha Shtraizent, Weehawken, NJ (US); Lina Freage, Weehawken, NJ (US); Henry Grage, Weehawken, NJ (US)

(21) Appl. No.: 18/982,589

(22) Filed: Dec. 16, 2024

Related U.S. Application Data
(60) Provisional application No. 63/651,554, filed on May 24, 2024.

(51) Int. Cl. C12Q 1/6825; C12Q 1/6876; G01N 21/64; G01N 33/74

(52) U.S. Cl. CPC C12Q 1/6825; C12Q 1/6876; G01N 21/6486; G01N 33/743; G01N 2333/575

(57) ABSTRACT

The present technology relates in general to the enhanced detection and quantitative analysis of one or more small molecules in a biological sample. In particular, the technology disclosed herein relates to the detection and analysis of biologically relevant molecules, hormones and other clinically relevant biomolecules. In one aspect, the systems and methods comprise contacting the biological sample with a chimeric reporter nucleotide probe, and determining the concentration of the small molecule in the biological sample.

### CROSS REFERENCE TO RELATED APPLICATIONS

This application claims priority to U.S. Patent Application No. 63/651,554 filed May 24, 2024, which is hereby incorporated herein in its entirety.

### FIELD OF THE INVENTION

The present technology relates in general to the enhanced detection and quantitative analysis of one or more small molecules in a biological sample. In particular, the technology disclosed herein relates to the detection and analysis of biologically relevant molecules, hormones and other clinically relevant biomolecules.

### BACKGROUND

Limitations and disadvantages of traditional detection systems and methods will become apparent to one of skill in the art, through comparison of such systems and methods with certain aspects of the technology set forth in the remainder of this disclosure, including with reference to the claims.

### BRIEF DESCRIPTION OF THE DRAWINGS

FIG. 1 is an illustration of the chimeric reporter nucleotide probe (CRNP) DNA linker system technology disclosed herein.

FIG. 2 is an example of a nanodrop reading using Progesterone conjugated to ALF at 11-position (P11-ALF or P11A).

FIG. 3 illustrates the progesterone small molecule and the progesterone-DNA-conjugate.

FIG. 4 illustrates two different hormones annealed with cAB for double stranded DNA-conjugates.

FIG. 5 illustrates simplified examples of two different avidin reporter systems.

FIG. 6 illustrates the inherent complexity in avidin reporter systems.

FIG. 7 illustrates an annealed probe/protein (i.e., PA-HRP).

FIG. 8 shows ELISA data of PA-HRP compared to P3A-Biotin.

FIG. 9 shows data for Single and double strand performance.

FIG. 10a shows CNRP stability at 4 C, room temp, and 40 C over 24 hours.

FIG. 10b shows CNRP vs commercial small molecule stability at 4 C, room temp, and 40 C over 8 days.

FIG. 11 shows Single and double stranded DNA performance post serum incubation (treated and untreated).

FIG. 12 shows the specificity of commercial conjugates (P3HRP, P3PB).

FIG. 13 shows Specificity of competitor (P3AB) vs P3PB.

FIG. 14 illustrates a Protein-DNA-Based Competitor (P3A-HRP).

FIGS. 15a-b illustrates signal enhancement in different matrices (serum and PBS).

FIG. 16 shows conjugate recognition data (CRNP vs Commercial P3HRP Conjugate).

FIG. 17 shows a schematic diagram of a competitive assay using embodiments of probes of the present invention.

FIG. 18 shows an embodiment of a competitive probe with a DNA linker.

### BRIEF SUMMARY

The current technology relates to systems and methods for the diagnostic analysis of small molecule antigens in a biological sample. In one aspect, the systems and methods comprise contacting the biological sample with a chimeric reporter nucleotide probe, and determining the concentration of the small molecule in the biological sample.

In a further aspect, the chimeric reporter nucleotide probe includes: (1) a nucleic acid-competitive antigen hybrid comprising a single stranded nucleic acid covalently attached to a competitive antigen; and (2) a nucleic acid-signaling reporter hybrid comprising a single stranded nucleic acid covalently attached to a signaling reporter.

In a still further aspect, the single stranded nucleic acid of the nucleic acid-competitive antigen hybrid is complementary to the single stranded nucleic acid of the nucleic acid-signaling reporter hybrid. Therefore, the single stranded nucleic acids of the nucleic acid-competitive antigen hybrid and the nucleic acid-signaling reporter hybrid base pair to form a double stranded duplex (—and thereby functionally link the competitive antigen and signaling reporter).

In another aspect of the technology described herein, there is provided a method for the diagnostic analysis of a small molecule antigen in a biological sample, the method comprising: contacting said biological sample with a chimeric reporter nucleotide probe; and determining the concentration of the small molecule in the biological sample.

In this aspect, the chimeric reporter nucleotide probe comprises: a nucleic acid-competitive antigen hybrid comprising a single stranded nucleic acid covalently attached to a competitive antigen; a nucleic acid-signaling reporter hybrid comprising a single stranded nucleic acid covalently attached to a signaling reporter; wherein the single stranded nucleic acid of the nucleic acid-competitive antigen hybrid is complementary to the single stranded nucleic acid of the nucleic acid-signaling reporter hybrid; and wherein the single stranded nucleic acids of the nucleic acid-competitive antigen hybrid and the nucleic acid-signaling reporter hybrid base pair to form a double stranded duplex in the chimeric reporter nucleotide probe.

### DETAILED DESCRIPTION

While the present technology will be described in connection with one or more preferred embodiments, it will be understood by those skilled in the art that the technology is not limited to only those particular embodiments. To the contrary, the presently described technology includes all alternatives, modifications, and equivalents as may be included within the spirit and scope of the appended claims.

Unless defined otherwise, all technical and scientific terms used herein have the same meaning as commonly understood by one of ordinary skill in the art to which the present disclosure belongs.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Generally, the present technology is directed to the enhanced detection and quantitative analysis of small molecules in a biological sample. The technology, in one aspect, includes contacting the biological sample with a chimeric reporter nucleotide probe, and subsequently determining the concentration of one or more small molecules in the biological sample.

The small molecule can be a hormone, including for example progesterone, estradiol, testosterone, androsterone, corticosterone, cortisone, cholesterol, estrone, estriol, or prednisoineor. The present technology relates to relates to probes that are easy to use and provide rapid results for detecting and quantifying levels of small molecules such as various hormones, including those generated via the endocrine system (e.g., adrenal glands, hypothalamus, ovaries, pancreas, parathyroid, pineal gland, pituitary gland, testes, thymus, and thyroid). These include, for example, the following classes: peptide hormones; steroid hormones; lipid derived hormones; and amino acid derived hormones.

The biological samples can be derived from various subjects, including human or non-human (e.g., agricultural and veterinary settings). For example, samples can be derived from women or men, at various age groups (e.g., pediatric, adult, elderly). Samples can be derived from subjects having a variety of different health status (i.e., healthy or sick) and at different stages of life (infancy, childhood, teenage, adult, elderly). The Samples collected generally include biological fluids, including for example interstitial fluids, blood, urine, serum, and saliva.

The technology described herein also has application in a number of settings, both clinical and non-clinical, including point-of-care. For example, clinical settings include general care settings, emergency care settings, labor and delivery care settings, elderly care settings, and pediatric care settings. Non-clinical settings include, for example, ambulatory settings, home care settings, work place settings, and battlefield settings.

The chimeric reporter nucleotide probe comprises: a nucleic acid-competitive antigen hybrid comprising a single stranded nucleic acid covalently attached to a competitive antigen; a nucleic acid-signaling reporter hybrid comprising a single stranded nucleic acid covalently attached to a signaling reporter; wherein the single stranded nucleic acid of the nucleic acid-competitive antigen hybrid is complementary to the single stranded nucleic acid of the nucleic acid-signaling reporter hybrid; and wherein the single stranded nucleic acids of the nucleic acid-competitive antigen hybrid and the nucleic acid-signaling reporter hybrid base pair to form a double stranded duplex in the chimeric reporter nucleotide probe.

The signaling reporter is preferably a fluorescent signaling molecule. In this aspect, the nucleic acid-signaling reporter hybrid can comprises multiple fluorescent signaling molecules, wherein the multiple fluorescent signaling molecules comprises a first fluorescent signaling molecule with high sensitivity, and a second fluorescent signaling molecule with a low (or lower) sensitivity. In another aspect, the multiple fluorescent signaling molecules comprises a first fluorescent signaling molecule with high sensitivity, a second fluorescent signaling molecule with intermediate (or medium) sensitivity, and a third fluorescent signaling molecule with low sensitivity.

The use of a chimeric reporter nucleotide probe provides significant enhancements in stability, user-friendliness, detection capabilities, sensitivity, specificity, and signal strength. In particular, the chimeric reporter nucleotide probes disclosed herein provide ease of use with high solubility in a wide range of buffers, low non-specific binding crucial for precise measurements in competitive ELISA, and the ability to measure probe concentration in the absence of reporter (e.g., using UV absorbance). Indeed, the chimeric reporter nucleotide probes disclosed herein provide exceptional stability under varied storage conditions and outstanding performance in ELISA assays, offering significant improvements in detection range, sensitivity, and variability compared to conventional protein-based conjugates. Still further, the chimeric reporter nucleotide probes disclosed herein provide versatility. In particular, the DNA linker system disclosed herein is based on a modular design that allows interchangeable components.

The DNA linker technology disclosed herein improves upon traditional conjugates by providing enhanced small-molecule performance and versatility. The increased adaptability allows for the swift integration of diverse probes and reporters into research and development processes. Importantly, the DNA linker technology imparts substantial mass to the small molecule probes, expanding their use in other biochemical applications such as lateral flow assays.

For many biochemical and biophysical applications (bioassays), including those for hormones that can affect clinical decisions, there exist needs to reduce variability and improve measurement accuracy. The present technology improves the variability in assay calibrations using novel conjugates that estimate endogenous hormone levels.

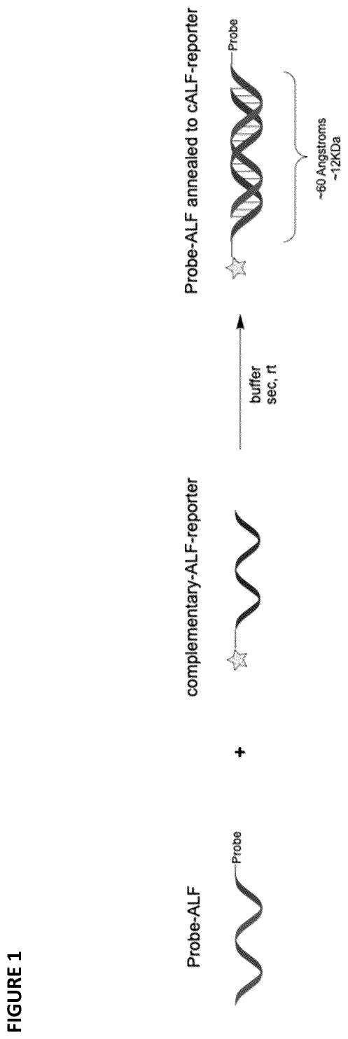

With conventional ELISA technology, conjugated probes (conjugates) compete against a set of calibrants for free antibody sites and report a signal that corresponds to a specific concentration. The present technology provides a DNA linker system, or chimeric reporter nucleotide probe (CRNP), that imbues a traditional conjugate with small-molecule-like performance and interchangeable pieces. As illustrated in FIG. 1, a probe is covalently attached to a single stranded (ss) DNA sequence (referred to as ALF). The complementary ALF sequence, referred to as cALF, is a piece of ss DNA bearing a reporter. The two single strands robustly anneal in standard buffers in a matter of seconds at room temperature.

The DNA linker system imparts interchangeability, and improves performance such as storage/stability, ease-of-use, wide ranges of detection, sensitivity, specificity, and signal strength. Additionally, the DNA imbues mass (12 KDa) onto small molecule probes that is useful in other biochemical applications such as Lateral Flow (LF).

### Ease of Use, Component Isolation, and Bioassay Integration

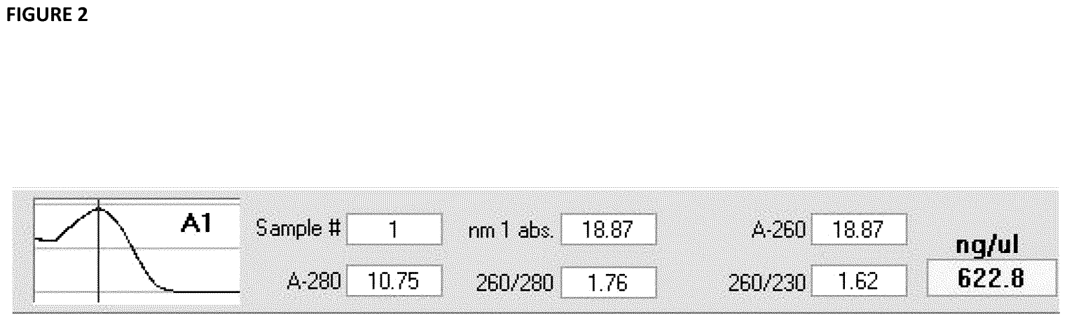

The DNA linker system provides easy and accurate means of quantification for small molecule probes and reporters, using standard DNA measurement methods such as nanodrop or Qubit (FIG. 2).

The CRNP utilizes DNA that naturally has high solubility in water and can make a poorly soluble probe dissolve in solution, making it compatible with common lab buffers. Indeed, the CRNP is easy-to-handle in routine lab practices using common disposable lab supplies.

Further, the present technology allows for the easy measurement of small molecule-DNA concentration. Reporter components can be added in excess to compensate for loss from non-specific binding and washed away without affection the competition ratio. What is more, a small molecule probe conjugated to DNA can be accurately measured and is the only concentration needed for a competitive approach. This avoids the issue of not being able to measure the concentration of DNA-proteins because of wavelength interference. Some reporter components are known to undergo non-specific binding that can affect the ratios of the components in competition. Non-specific binding can be troublesome to handle and lead to inconsistent results from experiment to experiment, also known as drift.

The ability to accurately measure concentration of the probe in the absence of the reporter component is a major advantage in competitive ELISA. The present technology allows for the easy measurement of small molecule-DNA concentration. Reporter components can be added in excess to compensate for loss from non-specific binding and washed away without affection the competition ratio.

### Interchangeable Components

Use of the interchangeable DNA linker is applicable to any suitable molecular system, not just hormones. Interchangeability allows for quick dynamic screening of appropriate reporter systems for research and development purposes, and permits quick adaptation and development of bioassays.

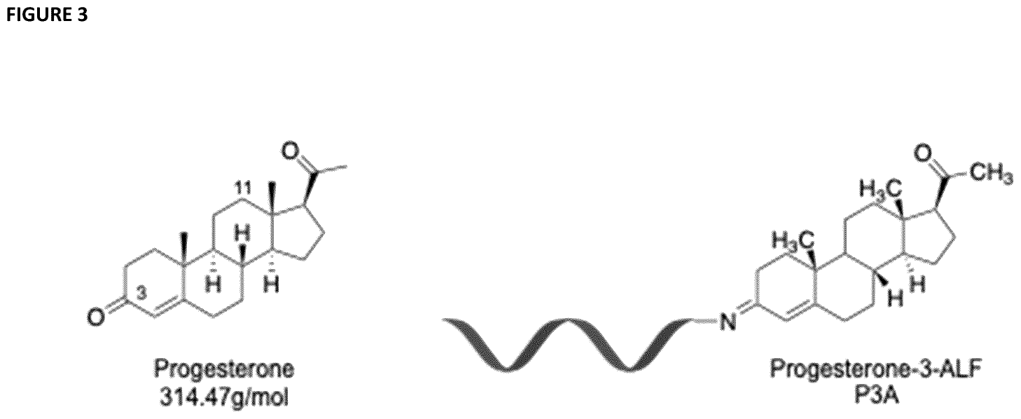

Many small molecule probes are linkable covalently to ALF, for example progesterone at the 3-position (P3A, FIG. 3).

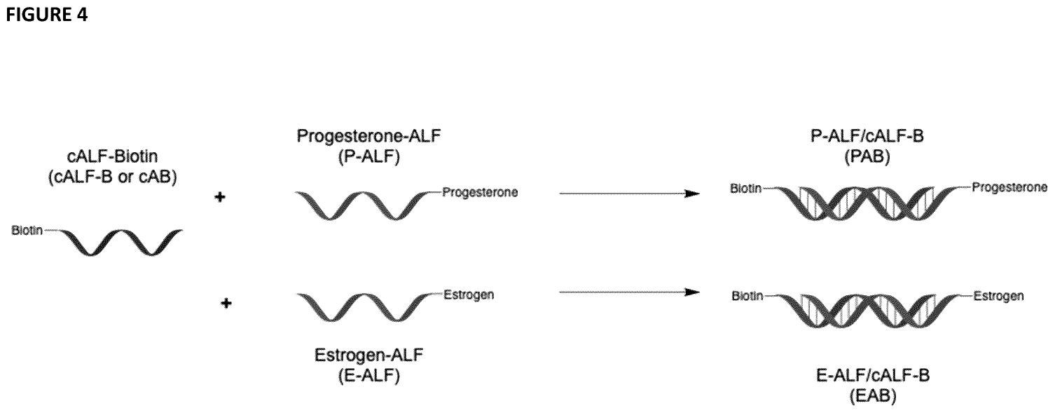

The annealed small molecule DNA conjugates may be used in separate hormone assays with the same cALF-reporter. For example, progesterone and estrogen annealed to cALF-biotin (cAB) is shown in FIG. 4.

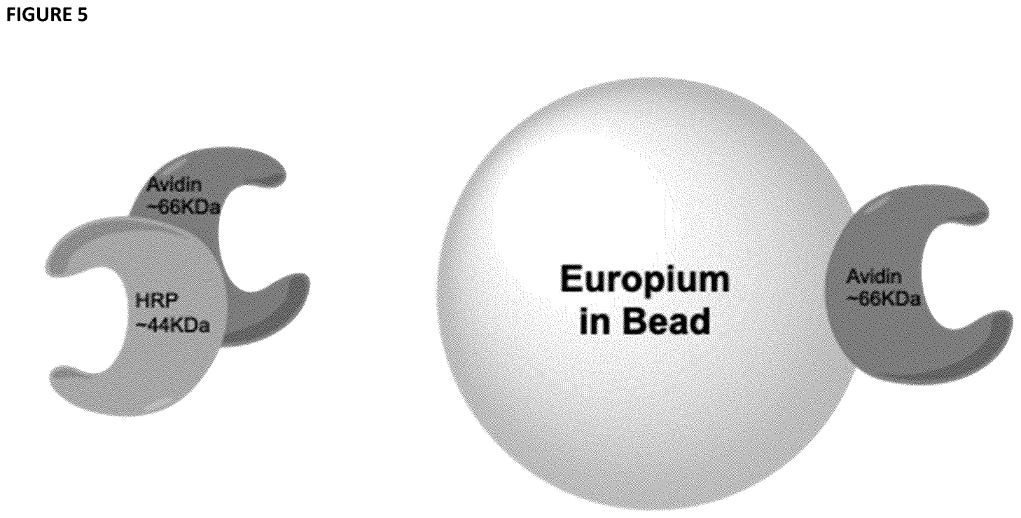

Also, the cALF may be directly conjugated to reporter systems, such as enzymes, fluorophores, and nanoparticles. cALF-Biotin can be employed for utility with widely available avidin reporter systems. For example avidin-HRP (colorimetric) and avidin-Europium Beads (fluorescence) can be used with hormone-DNA-Biotin, as illustrated in FIG. 5.

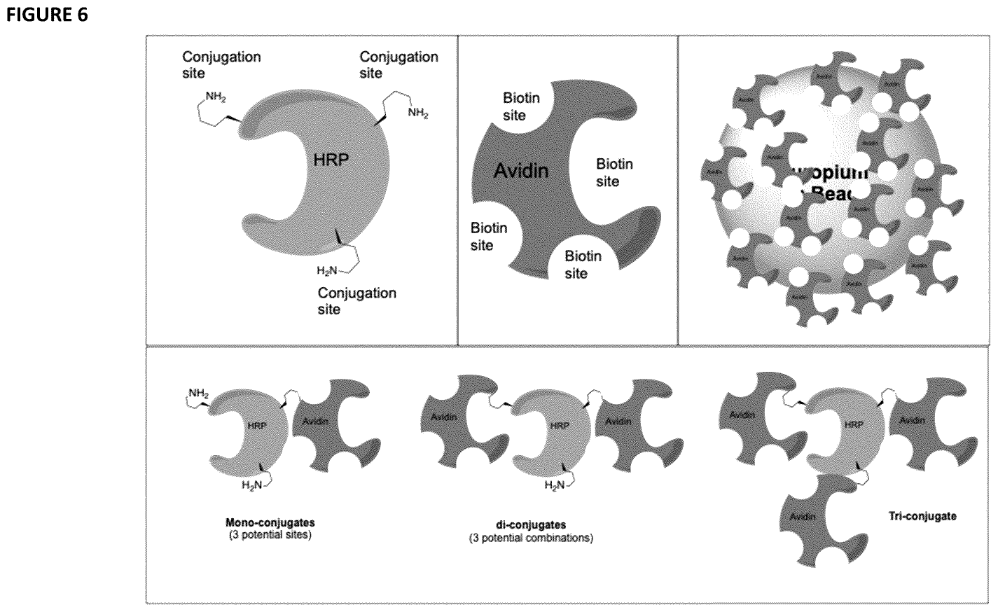

As illustrated in FIG. 6: HRP has 3 potential conjugation sites; avidin has 4 sites that recognize biotin; europium beads are covered with avidin proteins; and conjugation between HRP and avidin involves various sites that attenuate enzymatic activity and site recognition.

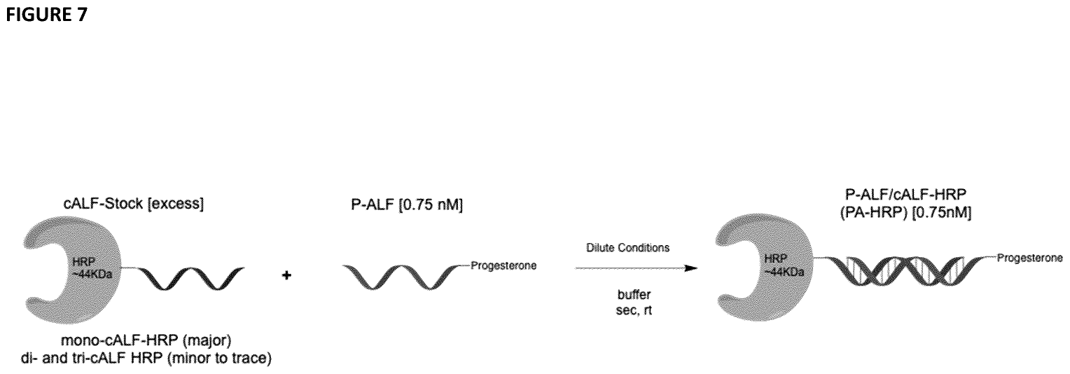

The interchangeability of the present technology accelerates and improves research and development. For example, as illustrated in FIG. 7, a cALF-horseradish peroxidase (cA-HRP) reporter modality can be quickly implemented into an ELISA format using the concentrations developed from the P3A-Biotin ELISA.

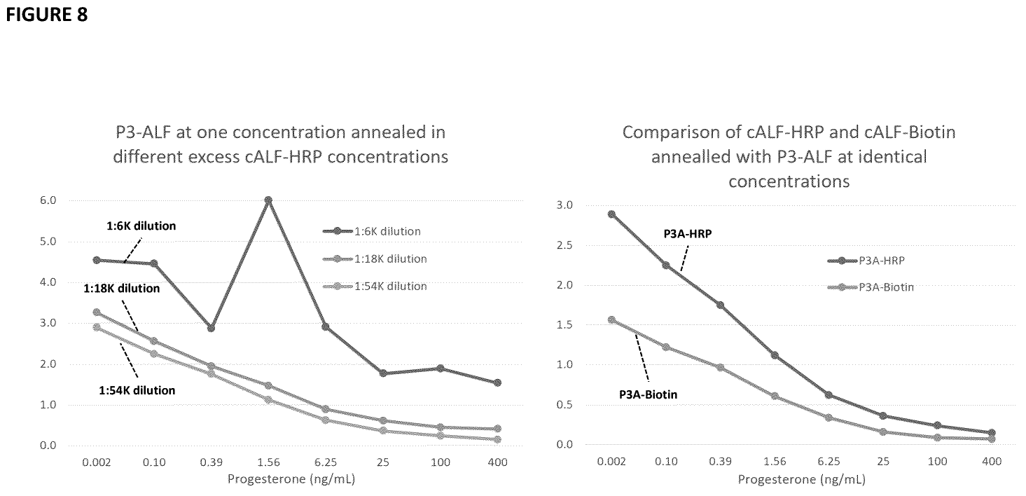

FIG. 8 illustrates PA-HRP ELISA data. As shown, cALF-HRP in high excess displays nonspecific binding to plate. The cALF-HRP in unknown excess to P3-ALF, P3A-HRP provides superior intensity performance to P3A-Biotin, which is more intense than P3P-Biotin.

### Versatility

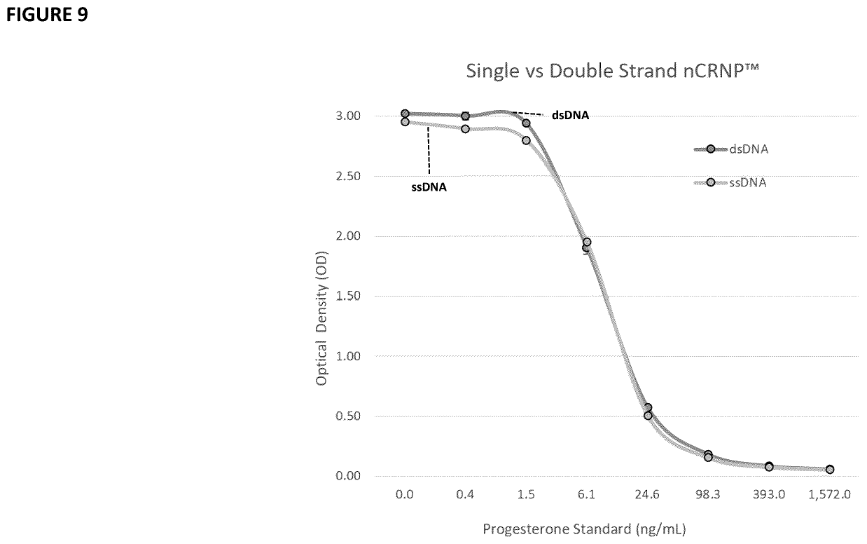

As illustrated in FIG. 9, single and double stranded competition are both viable in ELISA assays. This flexibility further allows individual assay optimization that may be required due to probe or reporter size and solubility.

### Stability

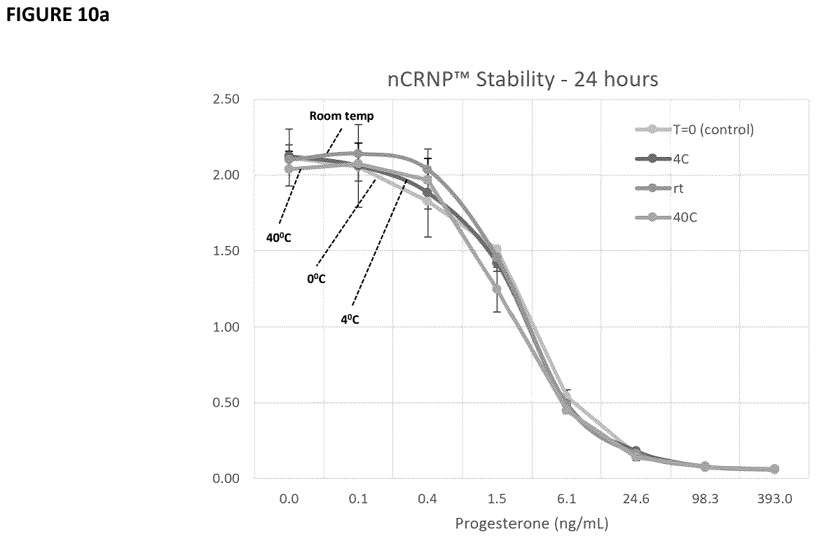

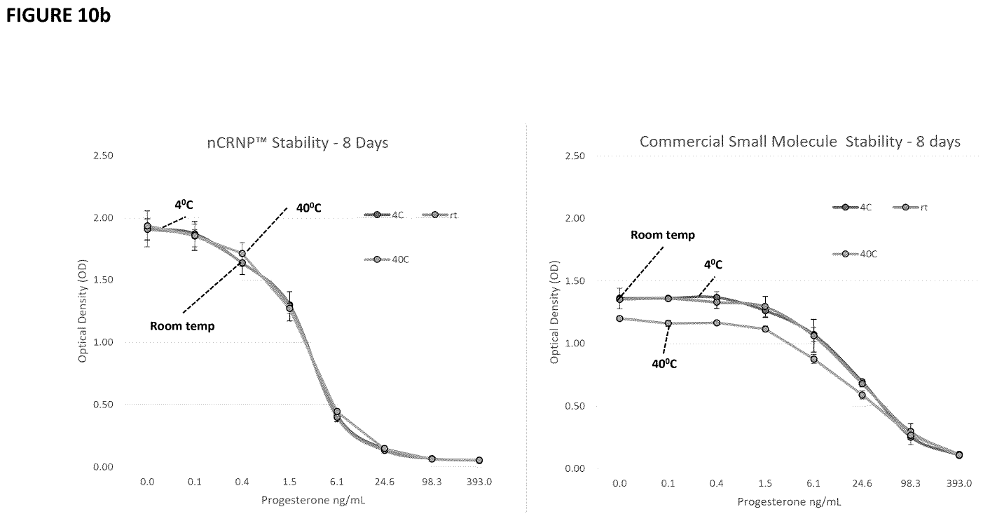

As illustrated in FIGS. 10a and 10b, the CRNPs disclosed herein have excellent stability in solution when stressed under differing storage conditions. The CRNP is stable to freeze/thaw periods. Also, there is no change in CRNP functionality in a Progesterone ELISA after 8 days when stored at different temperatures. Further, the CNRP was more stable than commercial small molecules even after 8 days at 40 C.

The CNRP can be stored as single or double stranded DNA. For example, working solutions were stored at 4 C in solution for at least one year without noticeable degradation. Storage conditions between the reporter molecule and the probe can vary greatly. Ideally, sensitive reporter components that require specialized storage or shipping conditions can be easily accommodated without involvement with the probe component.

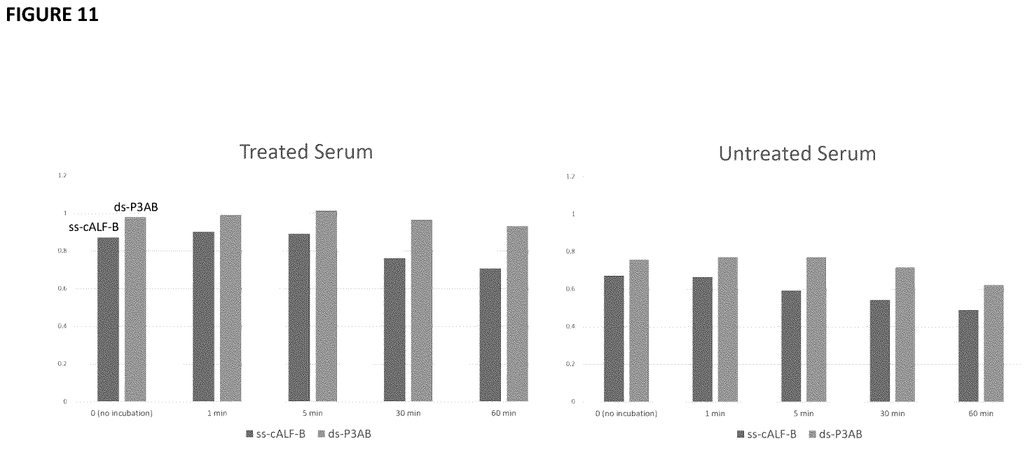

The single and double stranded DNA were incubated in treated and untreated serum samples. Preliminary data is illustrated in FIG. 11.

### Performance Comparison to FDA Approved ELISA Kits

In the ELISA format, the CRNP disclosed herein provide the several advantages over protein-based conjugates, including: wider range of detection; improved sensitivity; and less variability. As illustrated in Table 1, for progesterone the detection range increased 10-fold and sensitivity increased 4.5 fold.

TABLE 1

| | Reported Range | Sensitivity | |
|---|---|---|---|
| | (ng/mL) | (ng/mL) | Precision |
| Aveta. Life | 0.5-393 | 0.01 | <5% |
| DRG International | 0-40 | 0.045 | N/A |
| Diagnostic | 0.1-40 | 0.045 | 5.4-7% |
| International | | | |
| IBL America | 0.3-40 | 0.045 | 5.4-7% |
| Alpco | 0.3-60 | 0.1 | 10.2-10.6% |

As illustrated in Table 2, for estrogen, the sensitivity increased 3-fold against current ranges on the market and was able to separately increase detection range 3 to 5-fold.

TABLE 2

| | Reported Range | Sensitivity | |
|---|---|---|---|
| | (pg/mL) | (pg/mL) | Precision |
| Aveta - High Range | 1-10,000 | 28 | 3.8-7.9% |
| Aveta - Low Range | 1.5-3,333 | 3 | 0.6-5.5% |
| DRG International | 10.6-2,000 | 10.6 | 8.7-9.2% |
| Alpco | 20-3,200 | 10 | 4.6-9.3% |
| IBL American | 10.6-2,000 | 10.6 | 8.7-9.2% |

### Conjugate Specificity Towards Antibodies (Antibody Recognition): CRNP (P3AB) vs Commercial Conjugates in ELISA

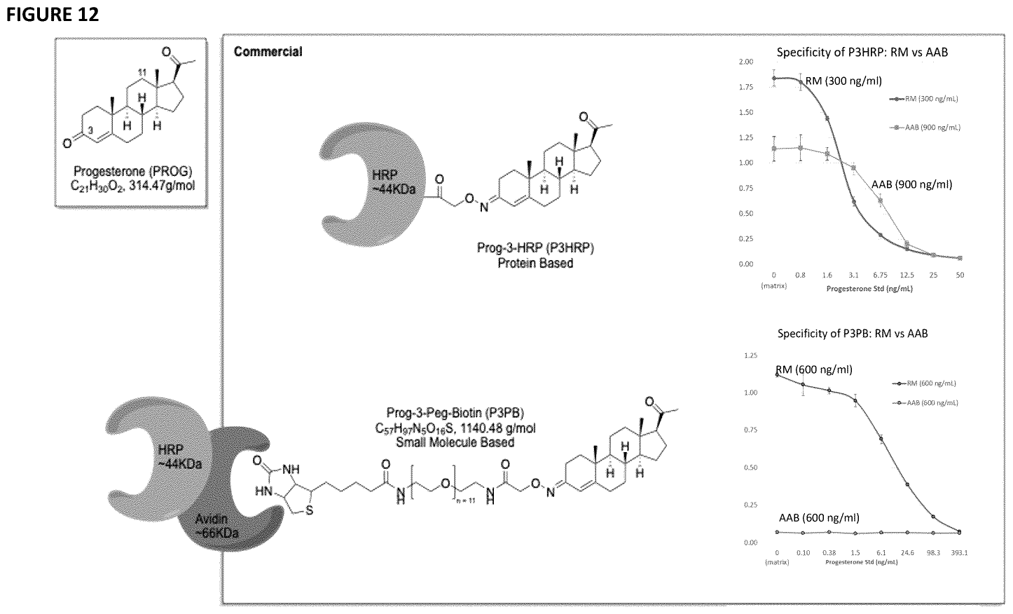

FIG. 12 shows the specificity of commercial conjugates (P3HRP, P3PB).

Protein based P3HRP conjugate is commercially available. The P3HRP handles poorly with troublesome non-specific binding to all plastic laboratory equipment, including expensive low binding coated plastic. Screening for a working concentration is performed by serial dilutions to find a working ratio, such as 1:80. P3HRP is recognized by two antibodies (RM and AAB).

Small molecule based P3PB (FIG. 12) is commercially available. The small molecule P3PB handles well without troublesome non-specific binding to laboratory equipment. P3PB is recognized only by the RM antibody and not recognized by the AAB antibody in these conditions. Avidin/HRP reporter is not present during the assay competition step, which implicates the HRP as a source of cross antibody specificity.

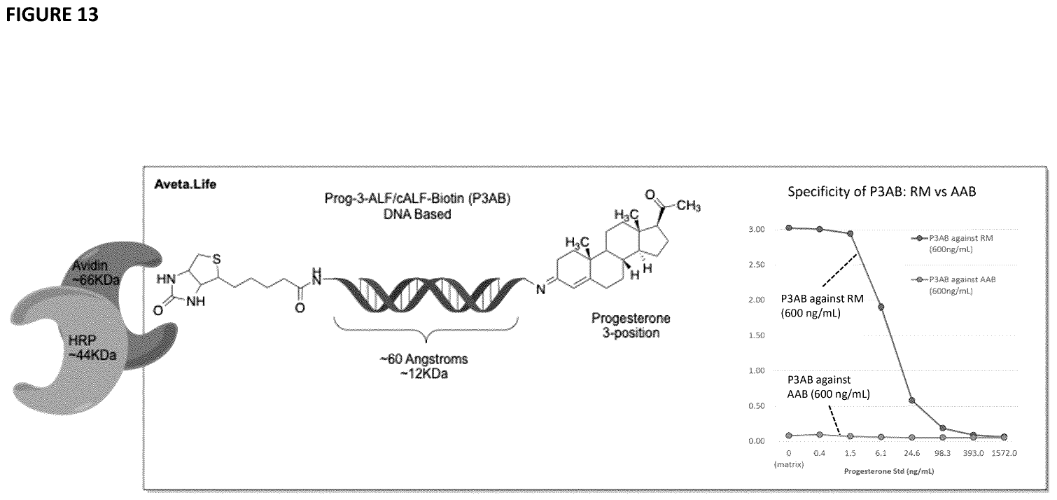

As illustrated in FIG. 13, P3AB performance is “small-molecule-like”. The CRNP handled well without non-specific binding to laboratory equipment. P3AB is recognized only by the RM antibody and not recognized by the AAB antibody in these conditions. While the DNA imbues mass, like the protein, no AAB recognition of the conjugate, confirms “small-molecule-like” performance in ELISA. Avidin/HRP reporter is not present during the assay competition step.

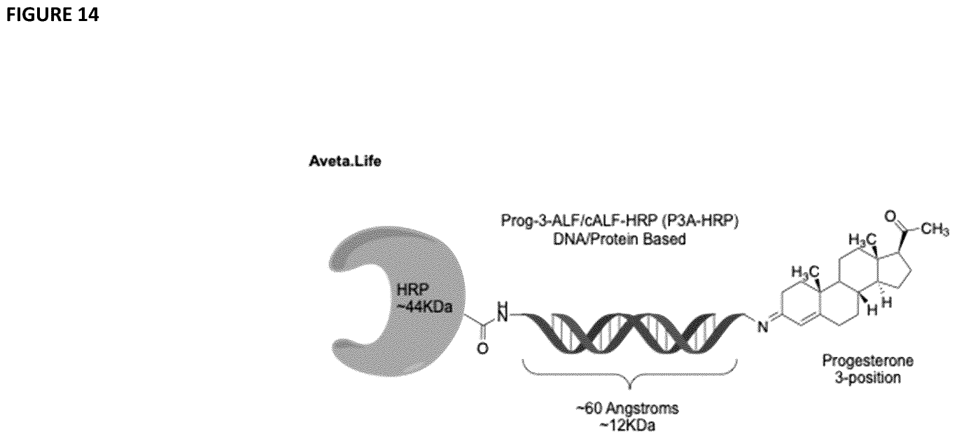

As illustrated in FIG. 14, c-ALF-HRP conjugate was prepared, annealed in excess to an accurate concentration of P-ALF, and used against the RM antibody.

### Comparison of CRNP conjugates vs Small Molecule Conjugates in ELISA

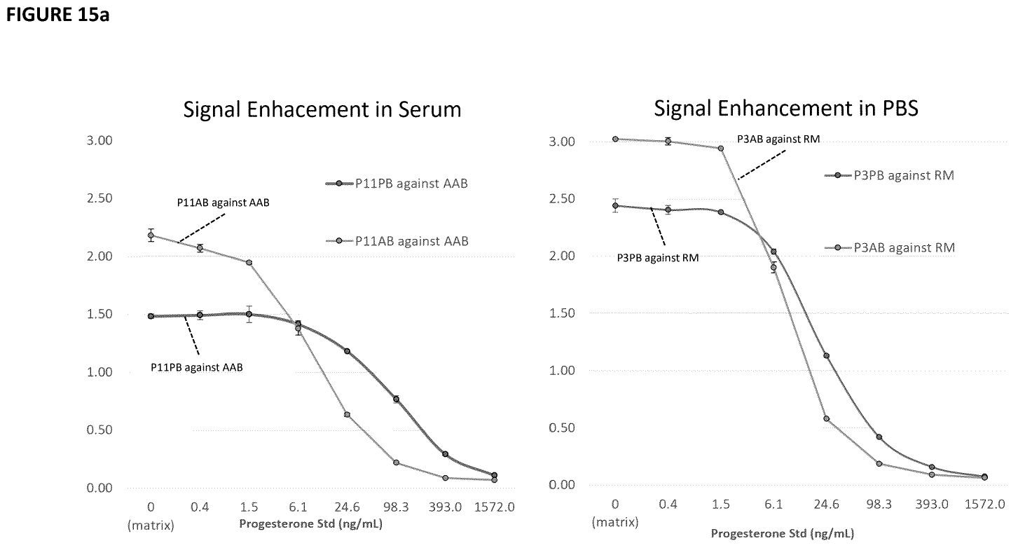

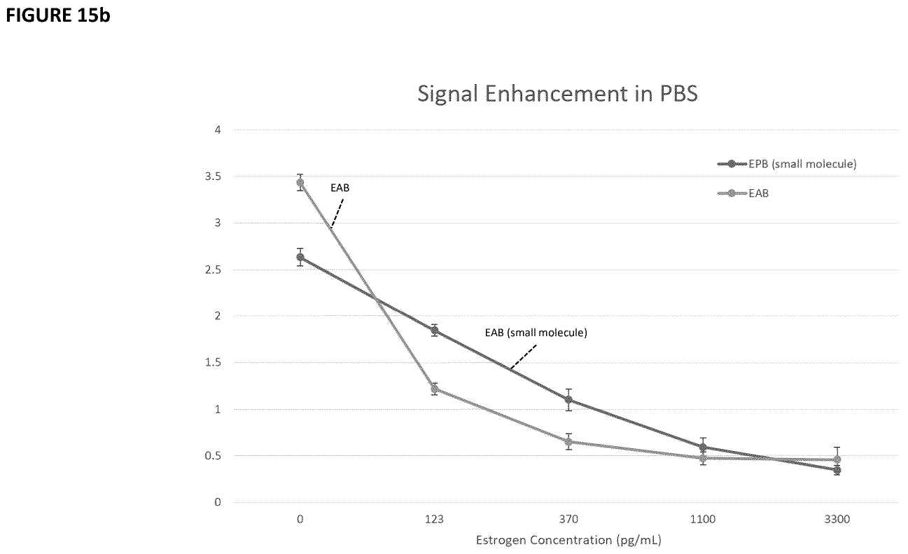

As illustrated in FIGS. 15a-b, CRNPs of the present technology provide the selectivity of small molecule based competitors with higher signal intensity than simple small molecules. Experiments done on the same plate and day show a higher signal with CRNP conjugates in different matrices. The CRNP DNA linker system provides 30% and 35% signal enhancement over small molecule competitors in aqueous and serum calibrations, respectively, with equal or less background signal.

### Conjugate Performance in the Presence of Potential Interfering Substances (Interference)

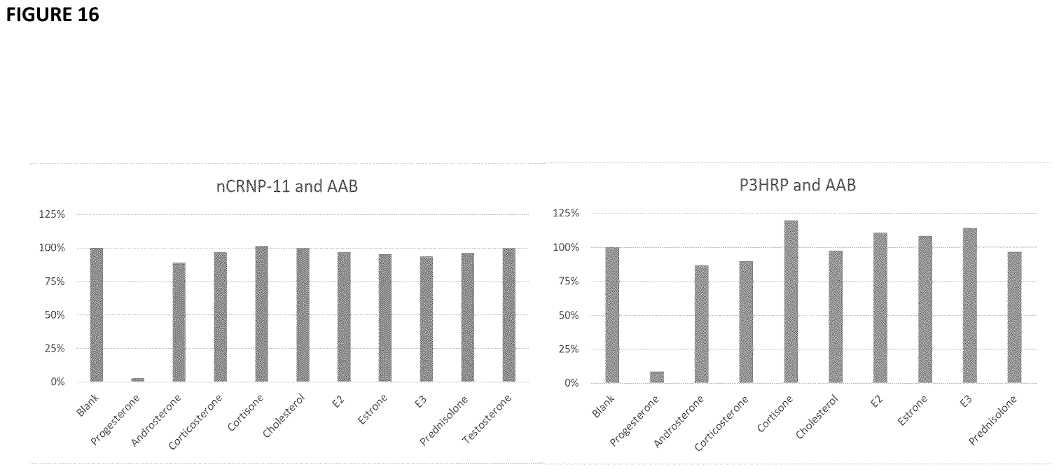

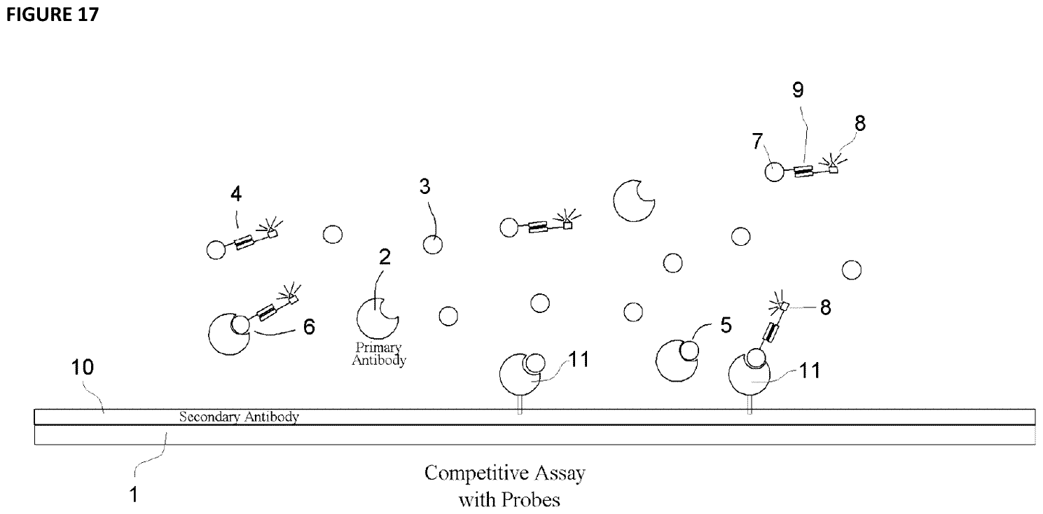

As illustrated in FIG. 16, P11AB provided a robust assay performance, higher top signal and sigmoidal shape vs P3HRP (commercial) against the AAB antibody. P11AB maintained performance in the presence of the potential interfering substances, as seen in our graph (FIG. 16, left). On the other hand, P3HRP performance was affected significantly by several substances (FIG. 16, right). The P3HRP provided less robust assay performance, including four signals higher than blank possibly to nonspecific binding events.

### General Overview

The technology disclosed herein includes probes that are easy to use and provide rapid results for detecting and quantifying levels small molecules such as various steroidal hormones like estrogen, progesterone and testosterone in samples.

The present invention provides generally a compound of the formula (I), or a salt, solvate, isotopically labeled derivative, stereoisomer, tautomer, or geometric isomer thereof, and any mixtures thereof having the structure:

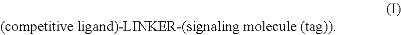

A competitive assay is shown in FIG. 1. The process takes place on a plate or other substrate 1. The plate is coated with a secondary receptor 10 such as an antibody. A receptor molecule 2 such as, but not limited to, a primary antibody sensitive to a particular small target molecule 3 such as a hormone is in solution near a plate, well or other substrate. The competitive ligand 7 is attached to a detectable tag 8 through a chemical and/or DNA linker 9. The competitive ligand binds 6 to a receptor 2 in solution so as to compete with the small target molecule 3. The bound complexes 11 then bind to the secondary molecule on the plate. The signaling molecule (tag) 8 allows detection, while the LINKER 9 is selected such that it allows for the compound to bind to a target and simultaneously facilitate detection. After flushing off unbound products, the detection signal is measured. A large signal results from a large number of bound probes which indicates a lower concentration of the target; a small signal results from a large number of bound target molecules which indicates a larger concentration of target molecules.

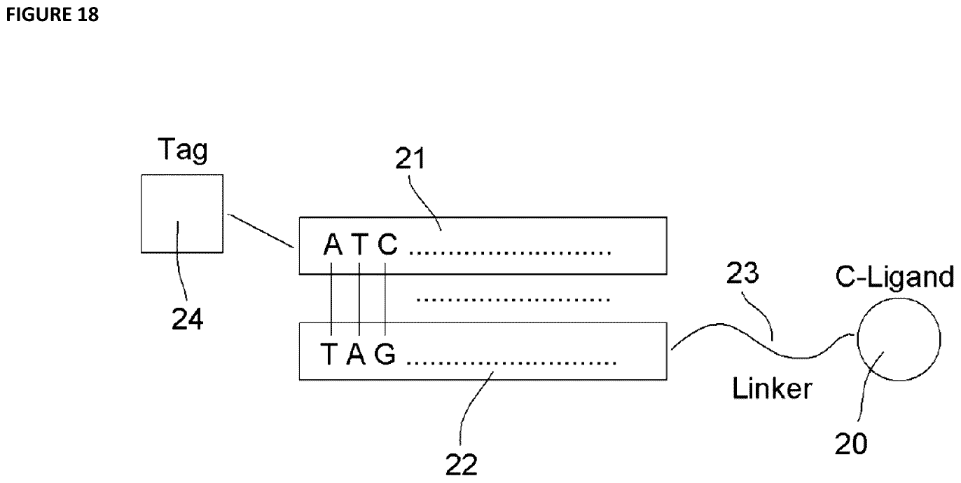

In an alternative embodiment shown in FIG. 18, the present invention provides a compound of the formula (II), or a salt, solvate, isotopically labeled derivative, stereoisomer, tautomer, or geometric isomer thereof, and any mixtures thereof that includes deoxyribonucleic acid (DNA):

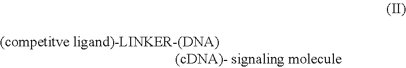

where—(DNA) represents a single-strand sequence of DNA 21 bound to a first compound such as the competitive ligand 20, and (cDNA)—22 is the single strand complement of the DNA 21 which is bound to second compound such as a signaling molecule 24. The competitive ligand 20 can be attached to the cDNA or DNA by a suitable chemical linker 23. The two DNA segments are linked typically linked together by hydrogen bonds

The DNA can be modified or unmodified nucleic acid (e.g., synthetic nucleotide analogs). The DNA can be from 10 to 15, from 10 to 20, from 10 to 25, from 10 to 30, from 10 to 40 nucleic acid bases. The DNA can have a GC content of from 30 to 60, from 35 to 55, or from 40 to 50 percent. The cDNA can be modified or unmodified nucleic acid. The cDNA can be from 10 to 15, from 10 to 20, from 10 to 25, from 10 to 30, from 10 to 40 nucleic acid bases. The cDNA can have a GC content of from 30 to 60, from 35 to 55, or from 40 to 50 percent. The DNA/cDNA duplex can have a melting points of between 40 to 65, 40 to 60, 40 to 55, 40 to 50, 45 to 55, 45 to 50° C.

### Selectivity and Cross-Reactivity

Precise Antigen Positioning: the rigidity of the DNA linker ensures that the antigen is presented in a specific orientation that is optimal for antibody recognition. This structured presentation minimizes the chances of non-specific binding to other molecules, enhancing the selectivity for the intended antigen.

Reduced Flexibility: the helical structure of the DNA linker restricts flexibility, ensuring that only antigens in the proper spatial conformation are exposed to the antibody. This reduces the likelihood of off-target interactions, as the linker prevents unwanted conformations that might encourage non-specific binding.

Minimized Crosstalk: by maintaining an appropriate distance between the antigen and other molecules (such as the reporter molecule), the DNA linker helps reduce crosstalk and interference from nearby molecular interactions. This spatial separation enhances selectivity by focusing the antibody's binding on the correct antigen without being influenced by surrounding elements.

Specific Binding Site Access: the rigid structure of the DNA prevents the antigen from folding or rotating into positions that may expose non-specific epitopes, ensuring that only the intended binding sites are available for antibody recognition. This enhances the precision of the interaction, improving selectivity.

The present invention should not be considered limited to the particular examples described above. Various modifications, equivalent processes, as well as numerous structures to which the present disclosure may be applicable, and which fall within the general scope of the disclosure, will be readily apparent to those of skill in the art to which the present disclosure is directed upon review of the instant specification.

### Sensitivity

Optimal Antigen Presentation: by maintaining a fixed, precise distance and orientation between the antigen and the antibody, the DNA linker ensures that the antigen is exposed to the most accessible conformation. This maximizes the likelihood of effective binding, as the antibody can more easily recognize and interact with the antigen, enhancing sensitivity.

Reduction of Steric Hindrance: the rigid helical structure of the DNA linker reduces steric hindrance, which could otherwise interfere with the antibody's ability to bind the antigen. This leads to a cleaner, more efficient interaction, contributing to improved sensitivity.

Minimized Background Noise: by keeping the reporter molecule and the antigen well-separated, the DNA linker minimizes interference from other molecules or elements in the system, reducing background signals and improving the overall sensitivity of detection.

What is more, as will be apparent to those of skill in the art upon reading this disclosure, each of the individual aspects described and illustrated herein has discrete components and features which may be readily separated from or combined with the features of any of the other several aspects, without departing from the scope or spirit of the present disclosure.

What is claimed is:

1. A method for the diagnostic analysis of a small molecule antigen in a sample, the method comprising:
contacting said biological sample with a chimeric reporter nucleotide probe; and
determining the concentration of the small molecule in the biological sample.

2. The method of claim 1, wherein the chimeric reporter nucleotide probe comprises:
a nucleic acid-competitive antigen hybrid comprising a single stranded nucleic acid covalently attached to a competitive antigen;
a nucleic acid-signaling reporter hybrid comprising a single stranded nucleic acid covalently attached to a signaling reporter;
wherein the single stranded nucleic acid of the nucleic acid-competitive antigen hybrid is complementary to the single stranded nucleic acid of the nucleic acid-signaling reporter hybrid; and
wherein the single stranded nucleic acids of the nucleic acid-competitive antigen hybrid and the nucleic acid-signaling reporter hybrid base pair to form a double stranded duplex in the chimeric reporter nucleotide probe.

3. The method of claim 1, wherein the small molecule antigen is a hormone.

4. The method of claim 1, wherein the small molecule antigen is progesterone, estradiol, testosterone, androsterone, corticosterone, cortisone, cholesterol, estrone, estriol, or prednisoineor.

5. The method of claim 2, wherein the signaling reporter is a fluorescent signaling molecule.

6. The method of claim 2, wherein the nucleic acid-signaling reporter hybrid comprises multiple fluorescent signaling molecules.

7. The method of claim 6, wherein the multiple fluorescent signaling molecules comprises a first fluorescent signaling molecule with high sensitivity, and a second fluorescent signaling molecule with low sensitivity.

8. The method of claim 6, wherein the multiple fluorescent signaling molecules comprises a first fluorescent signaling molecule with high sensitivity, a second fluorescent signaling molecule with medium sensitivity, and a third fluorescent signaling molecule with low sensitivity.

9. The method of claim 6, wherein the multiple fluorescent signaling molecules two different fluorescent signaling molecules having the same intensity.

10. The method of claim 1, wherein the sample is biological.
